(19) United States
(12) Patent Application Publication
(10) Pub. No.: US 2010/0149492 A1
(43) Pub. Date: Jun. 17, 2010

(54) Method for Determining an Ophthalmic Lens

(75) Inventors: Pascal Allione, Charenton-Le-Pont (FR); Diane De Gaudemaris, Paris (FR)

Correspondence Address: Hensley, Kim & Holzer, LLC, 1660 Lincoln St., Suite 3000, Denver, CO, 80264, US

(73) Assignee: ESSILOR INTERNATIONAL, Charenton Cedex (FR)

(21) Appl. No.: 12/520,865

(22) PCT Filed: Nov. 20, 2007
(86) PCT No.: PCT/FR07/01897

§ 371 Date: Feb. 11, 2010

(30) Foreign Application Priority Data
Dec. 22, 2006 (FR) 0611252

(51) Int. Cl. G02C 7/02; A61B 5/00

(52) U.S. Cl. 351/246; 351/177; 351/176

(57) ABSTRACT

The invention relates to a method for the optimisation determination of an ophthalmic lens that comprises the steps of: measuring parameters representative of the eye-head behaviour of the wearer; determining a central area on the lens having a diameter (D_{c}) that depends on the measured parameters representative of the eye-head behaviour; determining a peripheral area on the lens; optimising the lens when worn by the wearer by applying power and astigmatism target values in the central area and target values of a parameter different from the wearer's power in the peripheral area for given watching directions. The invention reduces the thickness of the lens and optimises the wearer's peripheral vision.

### CROSS-REFERENCE TO RELATED APPLICATIONS

This application is a national stage filing based upon international application no. PCT/FR2007/001897, filed 20 Nov. 2007 and published on 10 Jul. 2008 under international publication no. WO 2008/081086 (the '897 application), which claims priority to French application no. 0611252, filed 22 Dec. 2006 (the '252 application). The '897 application and '252 application are both hereby incorporated by reference as though fully set forth herein.

### BACKGROUND

Any ophthalmic lens intended to be held in a frame involves a prescription. The ophthalmic prescription can include a positive or negative power prescription as well as an astigmatism prescription. These prescriptions correspond to corrections enabling the wearer of the lenses to correct defects of his vision. A lens is fitted in the frame in accordance with the prescription and with the position of the wearer's eyes relative to the frame.

In the simplest cases, the prescription is reduced to a positive or negative power prescription. The lens is termed unifocal and has a rotational symmetry. It is simply fitted in the frame in such a way that the wearer's main viewing direction coincides with the axis of symmetry of the lens.

For any ophthalmic lens, the laws of the optics of ray tracings imply that optical defects appear when the light rays deviate from the central axis of any lens. These known defects which comprise, inter alia, a curvature defect or a power defect and an astigmatism defect can generically be called obliquity defects of rays. A person skilled in the art knows how to compensate for these defects. For example, EP-A-0 990 939 proposes a method for determining, by optimization, an ophthalmic lens for a wearer having an astigmatism prescription.

An ophthalmic lens comprises an optically useful central zone which can extend over the whole of the lens. Optically useful zone means a zone in which the curvature and astigmatism defects have been minimized in order to allow a visual comfort that is satisfactory for the wearer.

Generally, the optically useful zone covers whole the lens which has a diameter of limited value. However, in certain cases, a peripheral zone can be provided on the periphery of the ophthalmic lens. This zone is termed peripheral because it does not meet the conditions of prescribed optical correction and has significant obliquity defects. The optical defects of the peripheral zone are not harmful to the wearer's visual comfort because this zone is situated outside of the wearer's field of view.

There are different situations in which an ophthalmic lens may have such a peripheral zone. For example, when the lens has a significant diameter which can be required by the shape of the frame, for example an elongated frame with a high curving contour, or when the power prescription is high, the lens has a significant edge or centre thickness. A reduction this significant edge or centre thickness is desired. It is also possible to provide a peripheral zone intended to improve the wearer's peripheral vision. For example, distortion, chromatic aberrations, prismatic deviations or other optical parameters can be optimized in the peripheral zone to the detriment of the prescribed optical correction.

In the case of an ophthalmic lens intended to be fitted in a frame curved by 15° for example, the glass has a spherical or toric face with a high curvature (or base), between 6 diopters and 10 diopters, and a face calculated specifically to achieve the optimum ametropia correction for the wearer in the optical centre and in the field of view. For example, for the same front face, having the same curvature, the rear face is machined to ensure the correction according to the ametropia of each wearer. In the case of a negative lens, the high curvature of the front face leads to a great thickness of the glass on the edges. In the case of a positive lens, the high curvature of the front face leads to a great thickness of the glass in the centre in the case of a positive lens. These great thicknesses increase the weight of the lenses, which is detrimental to the wearer's comfort and makes the lenses unsightly. Moreover, for some frames, the edge thickness has to be limited to allow the glass to be fitted into the frame.

In addition, in the case of a strong prescription lens, the cut-out lens has a significant edge thickness on the nasal side for a hypermetropic positive lens and on the temporal side for a myopic negative lens. These extra thicknesses of the edges make it more complicated to fit the lens in the frame and make wearing the ophthalmic lenses heavier. For negative lenses, the edge thicknesses can be reduced by planing with a manual facette. A thinning of the lens can also be controlled by optical optimization. An aspherization or an atorization can be calculated, at least for one of the faces of the lens with high curvature, taking into account the conditions when the lens is worn compared with a lens of the same prescription with a low curvature, in order to reduce the centre and edge thicknesses of the lens with a high curvature.

Known solutions of optical aspherization or atorization are for example described in the documents U.S. Pat. No. 6,698,884, U.S. Pat. No. 6,454,408, U.S. Pat. No. 6,334,681, U.S. Pat. No. 6,364,481, U.S. Pat. No. 6,176,577, U.S. Pat. No. 5,825,454, EP-A-0 371 460, FR-A-2 638 246 or also WO-A-97 35224. These solutions propose to reduce the edge and/or centre thickness of the glasses of ophthalmic lenses with rotational symmetry by aspherizing or atorizing the whole area of a surface of the lens, generally the prescription surface.

The applicant filed a patent application on 28^{th} Sep. 2006 under number FR 06 08515 entitled “Method for determining an ophthalmic lens” the subject of which is a lens optimized so as to have a reduced centre or edge thickness. Such a lens has a central zone ensuring the correction prescribed for the wearer, a peripheral zone the curvature of which is determined in order to ensure the reduction in thickness and a connecting zone between the central and peripheral zones.

Known solutions for optimizing the peripheral vision are also described for example in the patent document U.S. Pat. No. 6,364,481.

Previously adopted solutions for minimizing the thicknesses of the lens or for optimizing certain optical parameters in peripheral vision optimize the optical performances of the lens over the whole surface of the lens and for the current needs of the wearers.

It has been found that each wearer has a different eye-head behaviour. In the last few years it has therefore been sought to customize ophthalmic lenses, in particular progressive lenses, in order to best satisfy the needs of each wearer.

Under the trade mark VARILUX IPSEO®, the applicant markets a range of progressive lenses, which are defined in relation to the wearer's eye-head behaviour. This definition is based on the fact that, to view different points at a given height in the object space, a wearer can move either his head or his eyes, and that the viewing strategy of the wearer is based on a combination of head and eye movements. The wearer's viewing strategy influences the perceived width of the fields on the lens. Thus, the more the wearer's lateral vision strategy involves a movement of the head, the narrower the zone of the lens scanned by the wearer's vision. If the wearer moved only his head in order to look at different points at a given height of the object space, his view would still pass through the same point of the lens. The product VARILUX IPSEO® therefore proposes different lenses, for the same ametropia-addition pair, as a function of the wearer's lateral vision strategy. It has also been found that the size and the shape of the frame modify the wearer's lens-eye behaviour. Therefore there exists a need to optimize the progressive ophthalmic lens for the type of frame chosen.

The U.S. Pat. No. 6,199,983, for example, proposes to customize a progressive lens as a function of the “lifestyle” of the wearer, for example taking into account the shape of the frame. Nikon® markets such a lens under the trade mark Seemax® a unifocal lens optimized as a function of the size and the shape of the frame.

The U.S. Pat. No. 7,090,348, for example, proposes customizing a progressive ophthalmic lens as a function of the wearer's eye-head behaviour. A starting lens is then chosen, the viewing points of which are determined as a function of the wearer's viewing strategy in order to identify the zones of the lens which are particularly used by the wearer. The optical performances of the lenses are then optimized for these zones.

A need still exists, however, for a unifocal lens which better satisfies the specific needs of each individual wearer, in particular for minimizing the thicknesses of the lens or for improving the peripheral vision.

### SUMMARY

Implementations described and claimed herein address the foregoing problems by providing a method for determining an ophthalmic lens customized for a given wearer. In one implementation, a method for determining an ophthalmic lens includes measuring parameters representing the wearer's head-eye behaviour; determining a central zone on the lens whose diameter depends on the parameters representative of the measured eye-head behaviour; determining a peripheral zone on the lens; and optimizing the lens under conditions when being worn by applying target power and astigmatism values in the central zone and target values of a parameter other than the power of the wearer in the peripheral zone for given viewing directions.

According to an embodiment, measuring parameters representative of the wearer's eye-head behaviour includes calculating a gain value. According to an embodiment, the gain value may be the ratio of the angle of the head to the viewing angle for a fixed point in a given viewing direction.

According to an implementation method, the diameter of the central zone is determined from the following relationship: D_{c}=30*(2−GA).

According to the embodiments, the target parameter in the peripheral zone is chosen from given distortion values, given chromatic aberration values, given prismatic deviation values and given glass thickness values.

Embodiments also relate to a customized ophthalmic lens optimized by the determination method according to the embodiments and a visual device including such a lens. Other technical advantages are readily apparent to one skilled in the art from the following figures, descriptions, and claims.

Other implementations are also described and recited herein.

### BRIEF DESCRIPTIONS OF THE DRAWINGS

FIGS. 1a and 1b illustrate example diagrammatic views of lenses each having a central zone and a peripheral zone, respectively, for an eye mover and a head mover.

FIG. 2 illustrates an example diagrammatic view of a connected face of an example lens.

FIG. 3 illustrates an example graph showing the wearer's optical power along the meridian of a lens according to a first embodiment optimized in terms of distortion for an eye mover.

FIGS. 4 and 5 illustrate maps of optical power and resulting astigmatism for the lens of FIG. 3.

FIGS. 6 and 7 illustrate maps of distortion for the lens of FIG. 3 and for a non-optimized lens of the same prescription, respectively.

FIG. 8 illustrates a graph showing the wearer's optical power along the meridian of a lens according to a first embodiment optimized in terms of distortion for a head mover.

FIGS. 9 and 10 illustrate maps of the optical power and resulting astigmatism for the lens of FIG. 8.

FIGS. 11 and 12 illustrate maps of distortion for the lens of FIG. 8 and for a non-optimized lens of the same prescription, respectively.

FIG. 13 illustrates an example graph showing the wearer's optical power along the meridian of a lens according to another embodiment, optimized in terms of thickness for an eye mover,

FIGS. 14 and 15 illustrate maps of optical power and resulting astigmatism for the lens of FIG. 13.

FIGS. 16 and 17 illustrate diagrammatic cross-sections for the lens of FIG. 13 and for a non-optimized lens of the same prescription, respectively.

FIG. 18 illustrates a graph showing the wearer's optical power along the meridian of a lens according to an embodiment, optimized in terms of thickness for a head mover.

FIGS. 19 and 20 illustrate maps of optical power and resulting astigmatism for the lens of FIG. 18.

FIGS. 21 and 22 illustrate diagrammatic cross-sections for the lens of FIG. 18 and for a non-optimized lens of the same prescription respectively.

### DETAILED DESCRIPTIONS

The embodiments described herein contemplate a method for determining an ophthalmic lens having a central zone optimized in terms of acuity according to the wearer's prescription and a peripheral zone optimized so as to improve a given parameter of the lens, such as its thickness or a peripheral vision characteristic such as distortion, prismatic effects, chromatic aberrations or others. According to the embodiments, the size of the central zone is determined as a function of the wearer's vision strategy and in particular as a function of his eye-head behaviour.

Thus, the embodiments propose to adapt the size of the optically useful central zone as a function of the wearer's eye-head behaviour, such that the optimization of the peripheral zone is maximum for a head mover and is not perceived as a discomfort for an eye mover.

The wearer's eye-head behaviour can be measured for example with the VisionPrint System™ developed by the applicant, or a similar device where eye-head coordination parameters are determined. These parameters can be those measured in order to define the lenses marketed under the trade mark VARILUX IPSEO®, namely a gain (GA) and a stability coefficient (ST).

The gain (GA) is a parameter which gives the proportion of the head movement in the total viewing movement in order to reach a target. The gain GA can be defined as the ratio of the head angle to the viewing angle for a fixed point in a given viewing direction. The gain has a value comprised between 0.00 and 1.00. For example, a gain value of 0.31 indicates an eye-head behaviour having a preponderant movement of the eyes. The stability coefficient (ST) is a parameter which indicates the stability of the behaviour, i.e. the standard deviation around the gain value. Most wearers are stable and the value of the stability coefficient (ST) is generally less than 0.15.

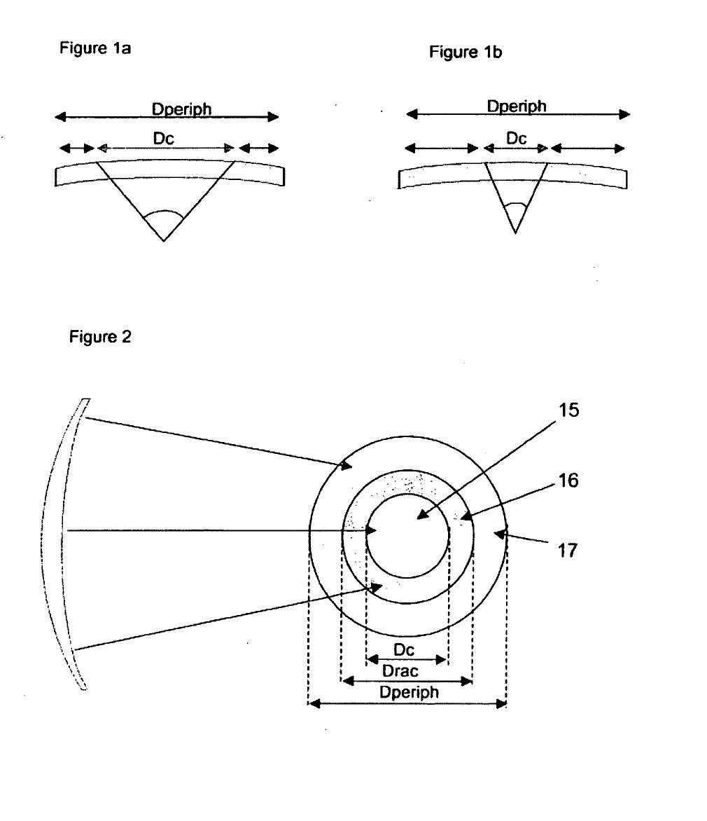

FIGS. 1a and 1b illustrate the modulation of size—or the diameter—of the central zone optimized in terms of acuity for a given wearer as a function of his eye-head behaviour. The central zone will thus have a relatively large diameter (D_{c}) when the wearer is an eye mover (FIG. 1a) and a relatively small diameter when the wearer is a head mover (FIG. 1b). In fact, when the wearer is an eye mover, he uses a large area of the glass whereas a wearer who is a head mover uses only a small area of the glass.

According to an implementation, the size of the central zone of the lens is fixed by choosing its diameter D_{c} as a function of the gain GA measured on the wearer. It is thus possible to construct a relationship for variation of the diameter of the central zone as a function of the wearer's eye-head behaviour which can be expressed as follows:

D_{c}=30*(2−GA) (1)

Thus, for a head mover (GA=1), a lens is obtained, where the central zone corresponding to the wearer's prescription is only 30 mm in diameter but with a peripheral border approximately 15 mm or more in width. This allows for satisfactory optimization of the thickness or of the peripheral vision. For an eye mover (GA=0), a lens is obtained where the central zone covers the whole surface of the lens. The optimization of the lens is calculated under the conditions in which it is worn for a lens diameter of 60 mm. For lenses with a larger diameter, the optimized peripheral zone is extrapolated.

The optically useful central zone and the peripheral zone must moreover be connected without discomfort for the wearer.

When the peripheral zone is optimized to improve the peripheral vision, the connection between the central and peripheral zones is made directly on the same surface when calculating the optimization of the prescription surface of the lens. When the peripheral zone is optimized in order to reduce the thickness, it is necessary to connect the central and peripheral zones by surface interpolation.

It is possible, for the connection of the central and peripheral zones in the case of lens thickness optimization, to use the method described in the abovementioned patent application filed by the applicant on 28^{th} Sep. 2006 under number FR 06 08515. In particular, the lens has a first face which can be spherical or toric, and a complex second face calculated to adapt the lens to the wearer's ametropia and to optimize the thickness of the lens under the conditions in which it is worn.

FIG. 2 illustrates a diagrammiatic view of a connected face of the lens. Opposite the spectacle wearer, a front surface is considered, which is spherical or toric having a maximum radius of curvature. A complex rear surface has three zones: an optically useful central zone 15 ensuring the correction necessary to the wearer in his field of view, a peripheral zone 17, and a connection zone 16 linking the central and peripheral zones. The central zone 15 may include a power and/or astigmatism correction, and its diameter D_{c} is fixed according to the abovementioned relationship (1) in order to take into account the wearer's eye-head behaviour. The surface of this complex rear face is continuous from a mathematical point of view and is machined just once by direct machining. The connecting zone 16 allows this mathematical continuity and ensures that the optical characteristics of the central zone 15 are not modified by the mechanical constraints imposed on the peripheral zone.

The three zones 15, 16 and 17 of the rear face are centred on the same point, preferably on the fitting cross which corresponds to the primary viewing direction of the wearer under the conditions in which the lens is worn. The three zones 15, 16 and 17 of the rear face of the lens have an identical shape, this shape (circular, elliptical, or other) being chosen according to the frame and/or the prescription. The dimension of the central zone 15 is imposed by the wearer's eye-head behaviour. The connecting zone 16 has to be wide enough to limit the visibility of the transition and narrow enough for the peripheral zone 17 to allow a particular optimization of the thickness.

The surfaces constituting the central zone 15 and peripheral zone 17 are known because they are imposed by the constraints of framing and/or prescription. The central zone 15 corresponds to the required power and astigmatism prescription. The central zone 15 can also be aspherized or atorized by means of an optical optimization. This aspherization/atorization can take into account the conditions in which the lens is worn, such as the curving contour angle and the pantoscopic angle of the frame. The calculation can also take into account a prismatic prescription allowing the effects of the curving contour and/or the pantoscopic angle to be corrected. The peripheral zone 17 can be a spherical or toric surface, according to the geometry of the front face. In the case of a spherical peripheral surface, the radius of curvature of the peripheral zone can be equal to the base of the front face; the lens is then flat in the peripheral zone. In the case of a toric peripheral surface, the meridian of largest curvature can be chosen equal to the base of the front face; the curvature value of the second meridian and the axis are chosen according to the lens prescription.

These surfaces of the central 15 and peripheral 17 zones are then sampled in a frame (X, Y, Z) associated with the rear surface of the lens. By convention, the X axis extends horizontally and the Y axis extends vertically when the lens is considered under the conditions in which it is worn. The Z axis is normal to the rear face of the lens. On the central zone 15 and peripheral zone 17, the altitude Z is known at each point (X, Y) of the surface. By convention, it is possible to fix the origin of the Z axis at the centre of the central zone 15. In this context, the altitude of the peripheral zone can be defined as the Z value at the lowest point of this zone, i.e. the minimum in terms of Z of the points situated on the circle of diameter D_{rac} delimiting the peripheral zone 17 towards the inside of the lens.

An interpolation formula calculates the altitudes Z of the points situated in the connecting zone 16 in order to define an interpolated surface which minimizes a merit function assessed for different relative altitudes of the peripheral zone compared with the central zone. The peripheral zone is therefore displaced in terms of Z until the interpolated surface which gives the smallest merit function is obtained. The Z-displacement of the peripheral zone does not alter the initial curvature characteristics of the central zone of the interpolated surface. The interpolated surface of the rear face can be calculated, for example, by a global spline interpolation method, as implemented in a MATLAB function (according to: de Boor, C., A Practical Guide to Splines, Springer-Verlag, 1978) or by a local polynomial interpolation method. The chosen merit function can be a minimization of the sphere or cylinder root mean square deviations calculated over a set of points, for example over the horizontal and vertical axes of the lens or over the circles of diameter D_{c} and D_{rac}, between the interpolated surface and the initial surfaces of the central and peripheral zones. The chosen merit function can also be a minimization of the cylinder value in the connecting zone 16 or a minimization of the sphere or cylinder slopes (norm of the gradient) in the connecting zone 16.

In order to carry out the optimization of a lens according to the present implementations, a lens having the required power and astigmatism prescription is considered as a starting lens.

A central zone having a diameter determined according to the above-mentioned relationship (1) is then defined. The size of the peripheral zone is also defined as a function of the desired optimization. If it is sought to optimize the lens in terms of thickness, a relatively broad peripheral zone is preferred on which maximum curvature radius criteria are imposed. For example, it is possible to impose substantially flat lens edges for optimum thinning of the lens.

The lens is considered under the conditions in which it is worn by setting the eye-lens distance q′, the pantoscopic angle (or vertical inclination) and curving contour values. The centre thickness of the lens and a lens index are provided. Targets are then are set for the optimization of the lens.

If the lens is optimized for the peripheral vision, it is possible, for example, to impose on the optically useful central zone targets having given power and resulting astigmatism module defect values for given viewing directions. Further, it is possible to impose on the peripheral zone targets having given distortion, chromatic aberration, prismatic deviation or other values. The lens is determined by optimization with the above targets.

If the lens is optimized to reduce its thickness, targets are fixed on the central zone, having given—preferably zero—power, astigmatism module and astigmatism axis values for given viewing directions. The lens is then determined by optimization by varying the characteristics of at least one face of the current lens so as to come close to the target values of the central zone while calculating an interpolated surface comprising a connecting zone between the central and peripheral zones. The interpolated surface can be calculated with a chosen interpolation formula and for a relative altitude of the peripheral zone compared with the given central zone. This relative altitude of the peripheral zone compared with the central zone is varied, i.e. the peripheral zone is moved away from or towards the central zone along the Z axis in order to obtain the best extrapolated surface compared with a given merit function, such as one of the merit functions mentioned previously—minimization of the sphere and cylinder root mean square deviations in the two directions X and Y or over the circles delimiting the central and peripheral zones; minimization of the maximum cylinder or the sphere or cylinder slopes in the connecting zone.

For the optimization, various representations of the surface or surfaces which vary can be used. The rear face and/or the front face of the lens can be varied. The face or faces which can be varied may be represented by Zernike polynomials. An aspherical layer, superimposed on one or other of the faces, may be used and this aspherical layer may be varied. The optimization can use techniques known per se. In particular, the damped least squares (DLS) optimization method can be used.

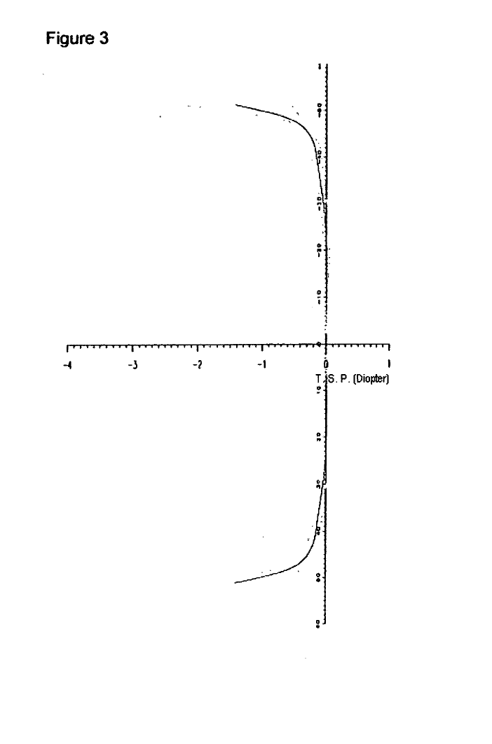

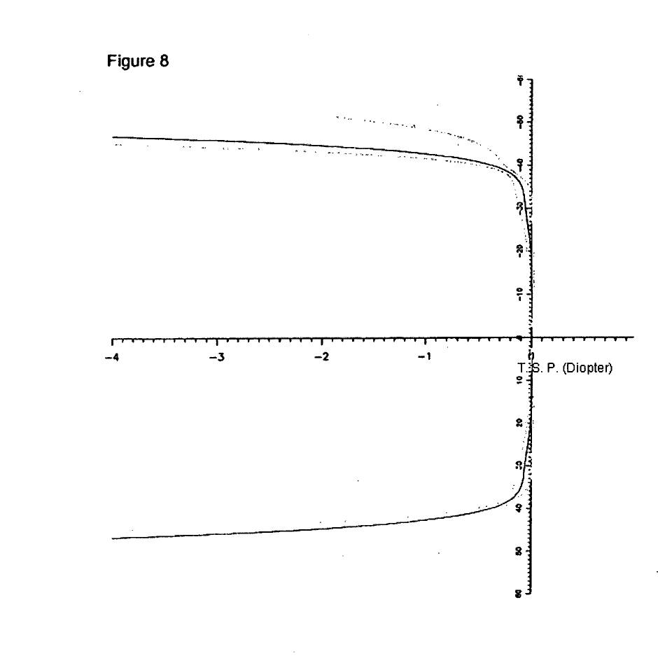

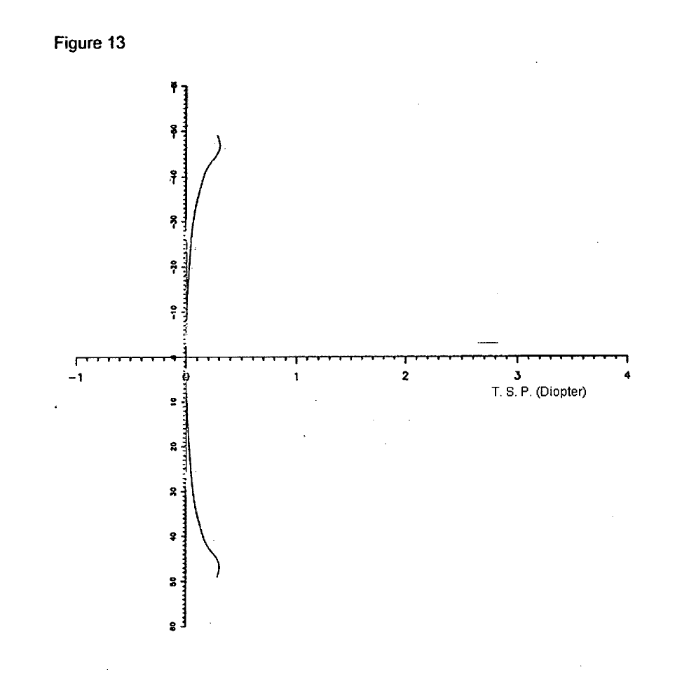

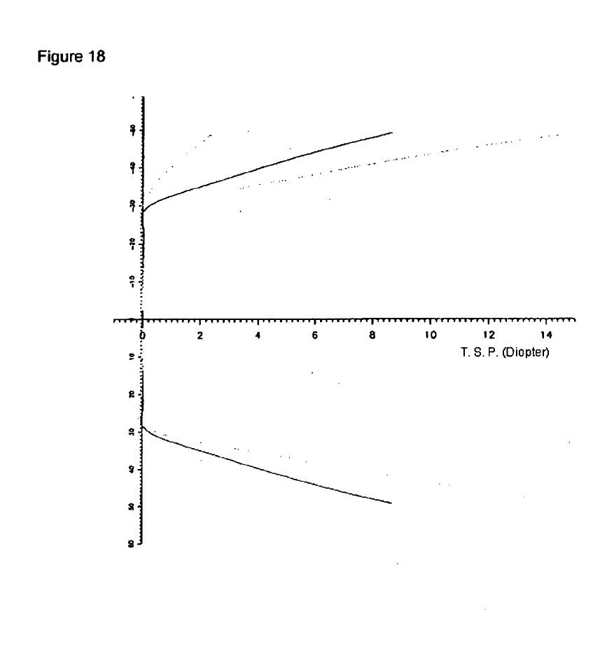

Lenses are described below with reference to several embodiments. FIGS. 3-7 illustrate an embodiment where the lens is optimized in terms of peripheral distortion for an eye mover. FIGS. 8-12 illustrate an embodiment where the lens is optimizes in terms of peripheral distortion for a head mover. According to another embodiment illustrated in FIGS. 13-17, the lens is optimized in terms of thickness for an eye mover. According to still another embodiment illustrated in FIGS. 18-22, the lens is optimized in terms of thicknessd for a head mover.

FIGS. 3 to 7 illustrate a unifocal lens with a total diameter of 60 mm and prescription of +3 diopters having a central zone suitable for an “eye mover” wearer for whom a gain of 0.33 has been measured. By applying the relationship (1) defined above, the central zone has a diameter of 50 mm. The peripheral zone is optimized in terms of distortion. The central zone is optimized in terms of acuity. The optical power is nearly constant and the resulting astigmatism is zero. FIG. 3 illustrates that the connection between the central zone and the peripheral zone introduces steps in power in the upper and lower parts of the meridian. However, these steps in power are situated beyond the wearer's natural field of view.

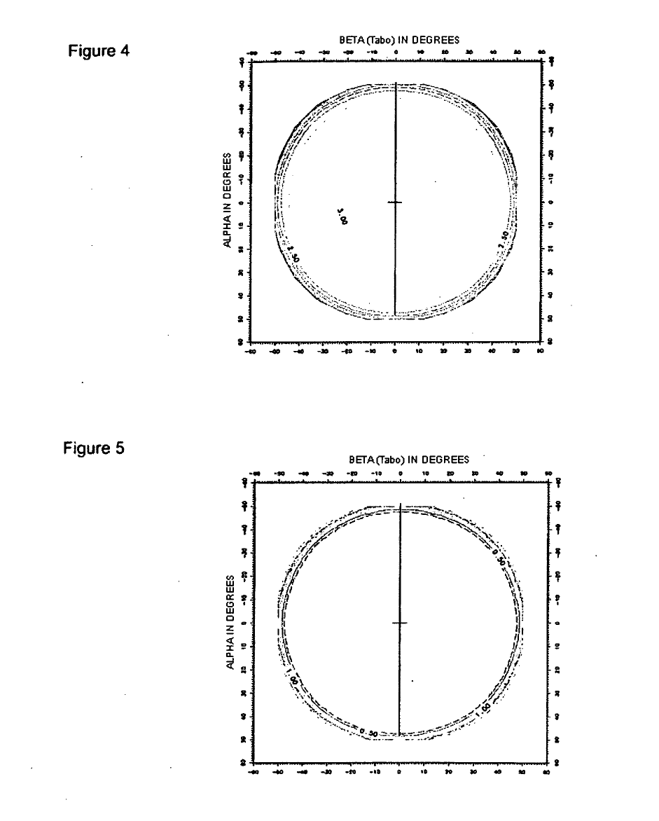

FIGS. 4 and 5 illustrate that the peripheral zone introduces power and astigmatism defects, but these defects are situated outside the wearer's natural field of view.

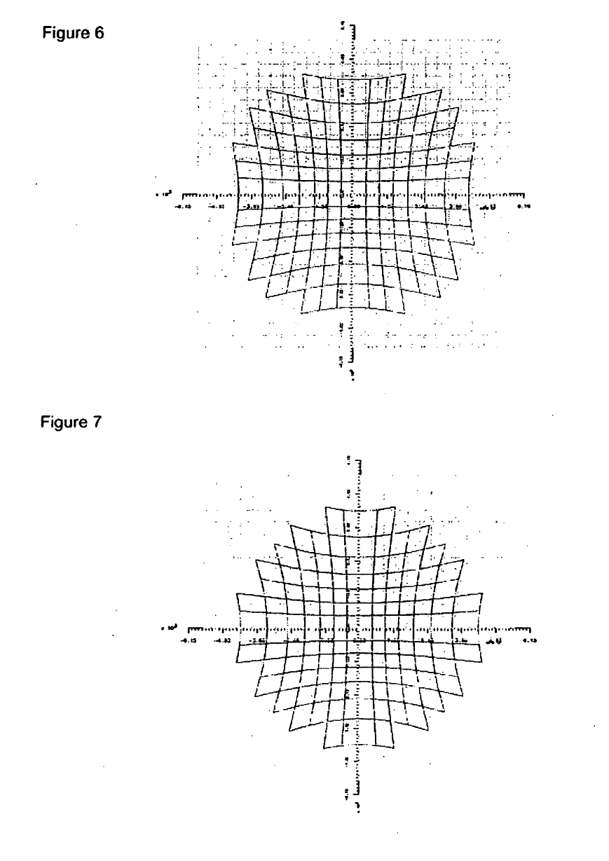

FIGS. 6 and 7 illustrate that the optimized lens provides an improvement in the distortion in the peripheral zone, at the same time improving perception in the wearer's peripheral vision and therefore his comfort. The distortion grids are identical in the central zone for the optimized lens and for a non-optimized lens. In contrast, the grid illustrated FIG. 6 (optimized lens) has less deformation at the periphery compared with the grid of FIG. 7 (non-optimized lens).

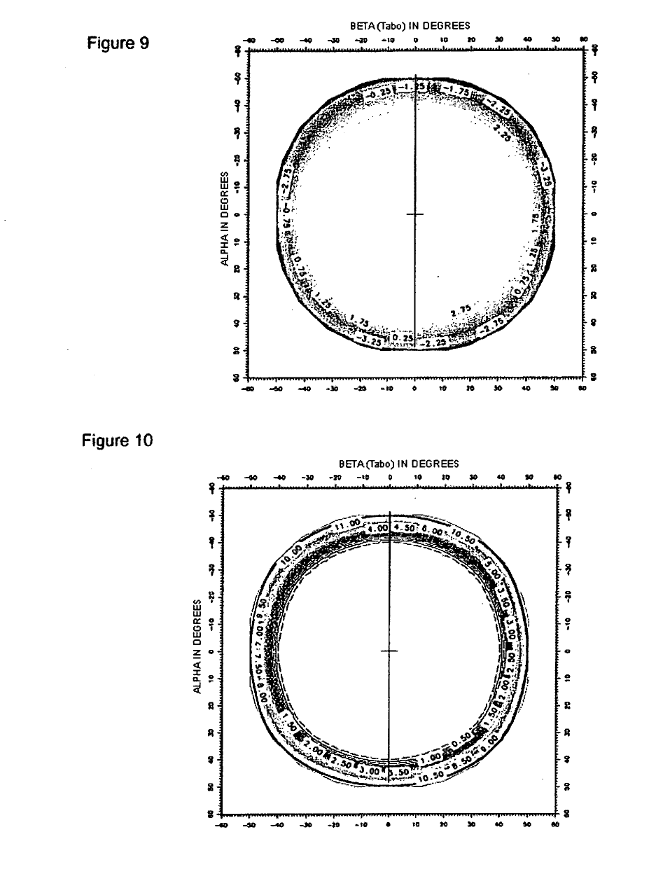

FIGS. 8 to 12 illustrate a unifocal lens with a total diameter of 60 mm and prescription of +3 diopters having a central zone suitable for a “head mover” wearer for whom a gain of 0.66 has been measured. By applying the relationship (1) defined above, the central zone thus has a diameter of 40 mm. The peripheral zone is optimized in terms of distortion. The central zone is optimized in terms of acuity; the optical power is nearly constant and the resulting astigmatism is zero. FIG. 8 illustrates that the connection between the central zone and the peripheral zone introduces steps in power in the upper and lower parts of the meridian. However, these steps in power are situated beyond the wearer's natural field of view. FIGS. 9 and 10 illustrate that the peripheral zone introduces power and astigmatism defects, but these defects are situated outside the wearer's natural field of view.

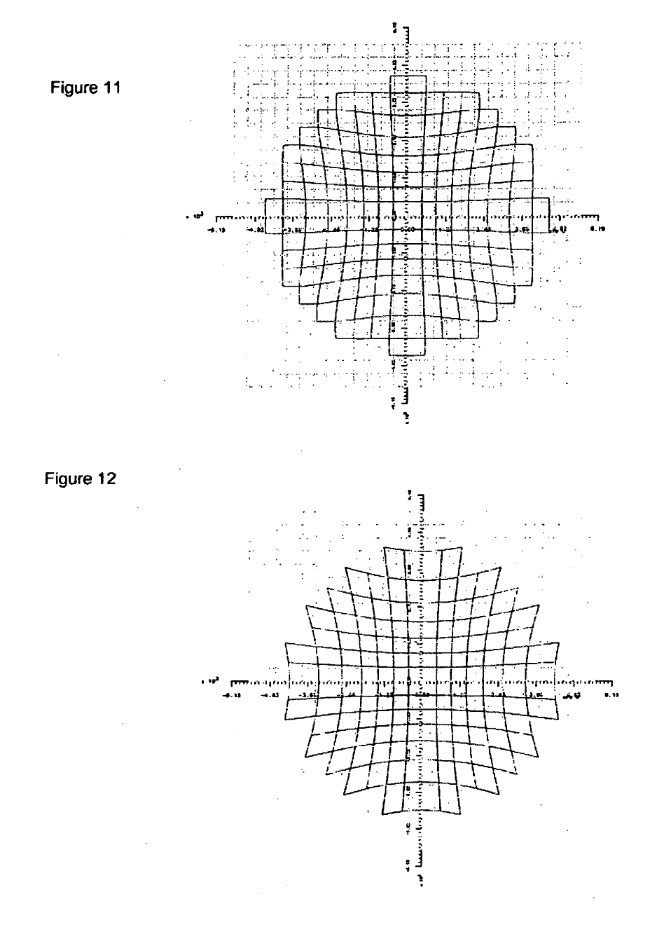

FIGS. 11 and 12 illustrate that the optimized lens provides a clear improvement of the distortion in the peripheral zone, at the same time improving perception in the wearer's peripheral vision and therefore his comfort. The distortion grids are identical in the central zone for the optimized lens and for a non-optimized lens. In contrast, the grid illustrated in FIG. 11 (optimized lens) has nearly no deformation at the periphery compared with the grid illustrated in FIG. 12 (non-optimized lens). The reduction of the distortion in the peripheral zone of the lens is more marked for the head mover (FIG. 11) than for the eye mover (FIG. 6) as the peripheral zone is larger and allows better optimization.

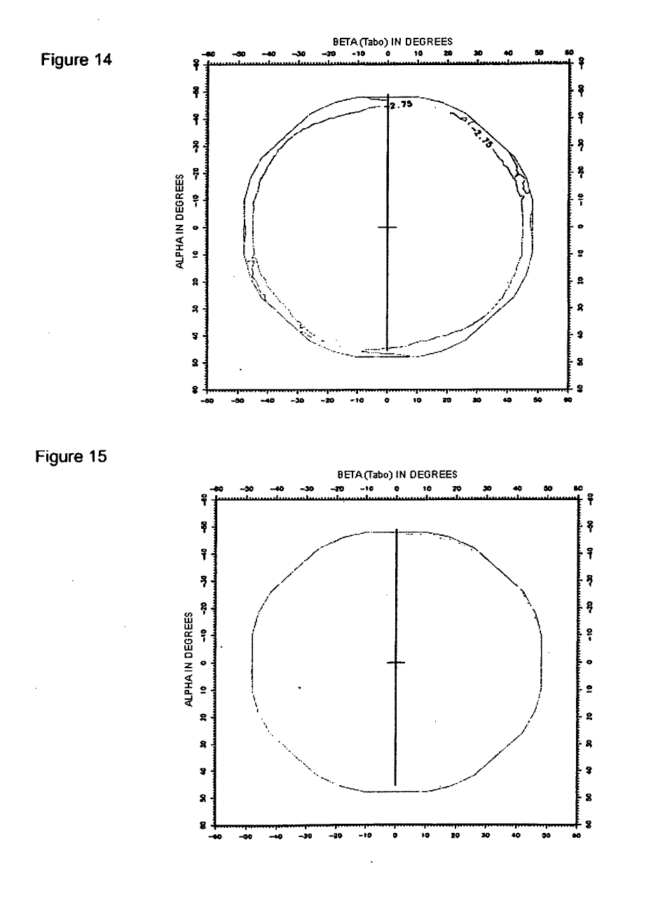

FIGS. 13 to 17 illustrate a unifocal lens with a total diameter of 80 mm and prescription of −3 diopters having a central zone suitable for an “eye mover” wearer for whom a gain of 0.33 has been measured. By applying the relationship (1) above, the central zone has a diameter of 50 mm. The peripheral zone is optimized in terms of thickness. The central zone is optimized in terms of acuity; the optical power is nearly constant and the resulting astigmatism is zero. FIG. 13 illustrates that the connection between the central zone and the peripheral zone introduces steps in power in the upper and lower parts of the meridian. However, these steps in power are situated beyond the wearer's natural field of view. FIGS. 14 and 15 illustrate that the connecting zone and the peripheral zone introduce significant power and astigmatism defects, but these defects are situated outside the wearer's natural field of view. These power and astigmatism defects are more marked than for the previous examples as the lens has a connected surface extrapolated with sphere values imposed on the peripheral zone in order to make the glass in the peripheral zone flat.

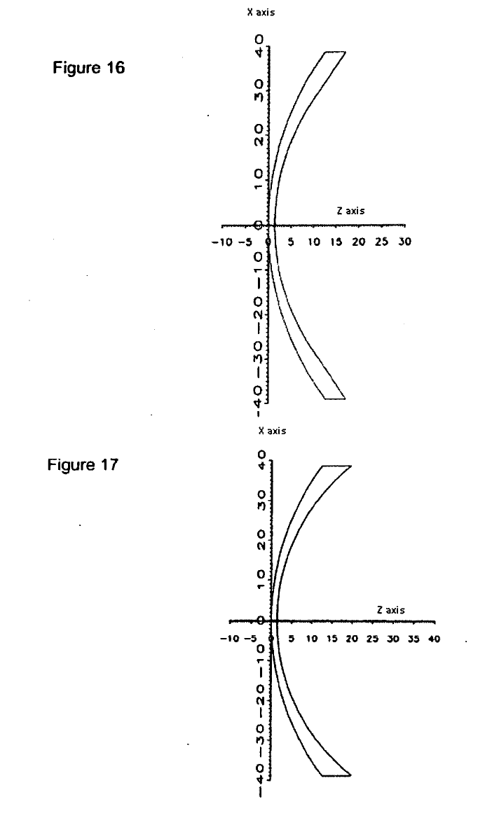

FIG. 16 illustrates a diagrammatic cross-section for the optimized lens. FIG. 17 illustrates a diagrammatic cross-section for a non-optimized lens of the same prescription and same dimensions. The standard lens (FIG. 17) has a centre thickness of 1.4 mm and an edge thickness comprised between 7.48 mm and 7.52 mm. In contrast, the optimized lens (FIG. 16) has a centre thickness of 1.4 mm for an edge thickness of 4.64 mm. Therefore implementations make it possible to considerably reduce the thickness of the lens. A lens thinned in this way is much lighter when worn and is easier to fit into a frame.

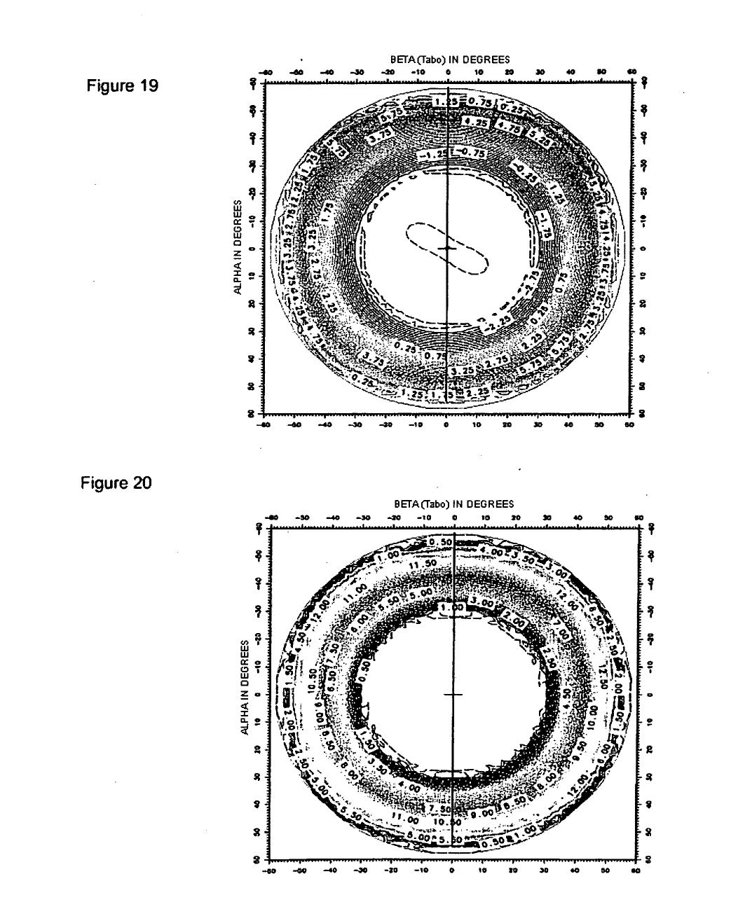

FIGS. 18 to 22 illustrate a unifocal lens with a total diameter of 80 mm and prescription of −3 diopters having a central zone suitable for a “head mover” wearer for whom a gain of 1 has been measured. By applying the relationship (1) defined above, the central zone thus has a diameter of 30 mm. The peripheral zone is optimized in terms of thickness. The central zone is optimized in terms of acuity; the optical power is nearly constant and the resulting astigmatism is zero. FIG. 18 illustrates that the connection between the central zone and the peripheral zone introduces steps in power in the upper and lower parts of the meridian. However, these steps in power are situated beyond the wearer's natural field of view. FIGS. 19 and 20 illustrate that the connecting zone and the peripheral zone introduce significant power and astigmatism defects, but these defects are situated outside the natural field of view of the wearer who uses only the central part of the lens.

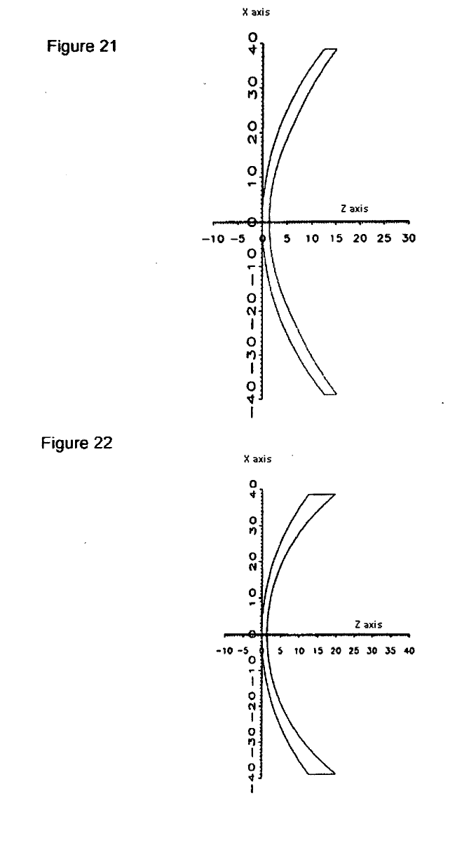

FIGS. 21 and 22 illustrate diagrammatic cross-sections of an optimized and a non-optimized lens, respectively, of the same prescription and same dimensions. The standard lens (FIG. 22) has a centre thickness of 1.4 mm and an edge thickness comprised between 7.48 mm and 7.52 mm. In contrast, the optimized lens (FIG. 21) has a centre thickness of 1.4 mm for an edge thickness of 2.67 mm. Therefore, implementations make it possible to considerably reduce the thickness of the lens, in particular for a head mover as the peripheral optimization zone is large. A lens thinned in this way is much lighter when worn and easier to fit into a frame.

The embodiments described herein may be implemented as logical steps in one or more computer systems. The logical operations of the present embodiments may be implemented (1) as a sequence of processor-implemented steps executing in one or more computer systems and (2) as interconnected machine or circuit modules within one or more computer systems. The implementation is a matter of choice, dependent on the performance requirements of the computer system implementing the embodiments. Accordingly, the logical operations making up the embodiments described herein are referred to variously as operations, steps, objects, or modules. Furthermore, it should be understood that logical operations may be performed in any order, unless explicitly claimed otherwise or a specific order is inherently necessitated by the claim language.

The above specification, examples, and data provide a complete description of the structure and use of exemplary embodiments. Since many embodiments can be made without departing from the spirit and scope of the invention, the invention resides in the claims hereinafter appended. Furthermore, structural features of the different embodiments may be combined in yet another embodiment without departing from the recited claims.

1-6. (canceled)

7. Method for determining an ophthalmic lens customized for a given wearer, comprising:
measuring parameters representing the wearer's head-eye behaviour;
determining a central zone on the lens whose diameter (D_{c}) depends on the parameters representative of the measured eye-head behaviour;
determining a peripheral zone on the lens; and
optimizing the lens under the conditions when being worn by applying target power and astigmatism values in the central zone and target values of a parameter other than the power of the wearer in the peripheral zone for given viewing directions.

8. A method according to claim 7, wherein measuring parameters representative of the wearer's eye-head behaviour includes calculating a gain value (GA).

9. A method according to claim 8, wherein the gain value is a ratio of the angle of the head to the viewing angle for a fixed point in a given viewing direction.

10. A method according to claim 8, wherein the diameter of the central zone is determined from the following relationship: D_{c}=30*(2−GA).

11. A method according to claim 7, wherein the target parameter in the peripheral zone is chosen from given distortion values, given chromatic aberration values, given prismatic deviation values, and given glass thickness values.

12. A method according to claim 7, wherein the target parameter in the peripheral zone is chosen from given distortion values, given chromatic aberration values, given prismatic deviation values and given glass thickness values, and
wherein measuring parameters representative of the wearer's eye-head behaviour includes calculating a gain value (GA).

13. A method according to claim 12, wherein the gain value is a ratio of the angle of the head to the viewing angle for a fixed point in a given viewing direction.

14. A method according to claim 7, wherein the target parameter in the peripheral zone is chosen from given distortion values, given chromatic aberration values, given prismatic deviation values and given glass thickness values,
wherein measuring parameters representative of the wearer's eye-head behaviour includes calculating a gain value (GA), and
wherein the diameter of the central zone is determined from the relationship: D_{c}=30*(2−GA).

15. A method according to claim 14, wherein the gain value is a ratio of the angle of the head to the viewing angle for a fixed point in a given viewing direction.

16. An ophthalmic lens customized for a given wearer, said lens having a central zone whose diameter is determined as a function of parameters representative of the eye-head behaviour measured on the wearer.

17. A visual device comprising a frame chosen by a wearer and at least one lens according to claim 16.
